(19) United States
(12) Patent Application Publication
(10) Pub. No.: US 2026/0345973 A1
(43) Pub. Date: Nov. 5, 2026

(54) ARTIFICIAL LYMPHATIC VESSEL FOR SECONDARY LYMPHEDEMA

(71) Applicant: CENTROS CULTURALES DE MÉXICO, A.C., Ciudad de México (MX)

(72) Inventors: Gabriela DURÁN AGUILAR, Zapopan, Jalisco (MX); Luis Alberto ROSA SIERRA, Zapopan, Jalisco (MX); Rita Quetziquel FUENTES AGUILAR, Zapopan, Jalisco (MX)

(21) Appl. No.: 19/118,852

(22) PCT Filed: Oct. 23, 2023
(86) PCT No.: PCT/IB2023/060697

§ 371 Date: Apr. 7, 2025

(51) Int. Cl. A61M 27/00; A61M 39/24

(52) U.S. Cl. CPC A61M 27/002; A61M 39/24; A61M 2202/0405; A61M 2206/20; A61M 2210/1007

(57) ABSTRACT

The present invention relates to an “artificial” lymphatic vessel that makes it possible to restore the circulation of lymph to the lymph nodes in cases where reconstruction is not possible, comprising a tubular body that has at least one inlet end with a first diameter and at least one outlet end with a second diameter. A one-way valve is arranged near the at least one outlet end, wherein the one-way valve comprises a sudden reduction of the first diameter to produce a third diameter, followed by an area of uniform reduction of the third diameter until reaching a discharge area with a fourth diameter.

### SUBJECT MATTER OF THE INVENTION

The general objective of this application is to obtain a lymphatic vessel as an internal medical device that may represent an option in restoring lymph circulation, which has been affected by treatments for severe and serious stages of breast cancer due to damage to the lymphatic vessels in cases where reconstruction is not possible.

### BACKGROUND

Breast cancer treatment requires a multidisciplinary approach, with surgery playing a fundamental role. The breast is treated with conservative or total surgery. Lymph node dissection is required to check the extent of the disease; this can be selective or radical, and determines what treatment the patient should receive, since treatment must be totally personalized.

Through studies by the American Cancer Society, it has been observed that lymphedema occurs during some of cancer surgery procedures. The doctor could remove lymph nodes near the tumor to determine if the cancer has spread. When this occurs, the lymphatic vessels that carry fluid from that area to the rest of the body are also eliminated, because they pass through the nodes and are connected around them. Removing lymph nodes and vessels makes it difficult for lymphatic fluid to flow from the arms, legs, and other parts of the body to the chest, where it is released back into the bloodstream. If the remaining lymph vessels cannot take up enough fluid from the area, fluid builds up and causes swelling, or lymphedema.

Another treatment that may cause lymphedema is radiotherapy, which can affect the transport of lymphatic fluid by causing damage and scar tissue in lymph nodes and vessels. Sometimes, the tumor itself can block part of the lymphatic system and cause lymphedema, as well as infections that limit the passage of lymphatic fluids. There is no foolproof way to prevent all cases of lymphedema; however, there are ways to reduce the risk of developing the condition. For example, when lymph nodes need to be removed, some modern surgical techniques can be useful. The doctor may use a procedure selecting only the infected nodes, avoiding the previous methods of dissecting all the nodes without knowing which one was sick. With this type of method, the removal procedure is safer and less aggressive for the patient.

Based on the function of the lymphatic system, which is made up of lymphatic vessels and organs, the interstitial fluid is reabsorbed and transported in vessels of increasing caliber to the pre-collector and collector. The lymph nodes filter lymph and prevent pathogens from entering the bloodstream; there are two compartments within the lymphatic system for this purpose:
1.—The superficial nodes drain the skin
2.—The deep nodes drain the lymph produced in the muscles, bones, joints and nerves.

The present invention is intended solely to resolve secondary lymphedema. Therefore, it focuses on curing and not only on treating the symptoms. However, the concepts of lymph, lymph nodes and lymphatic vessels must first be presented as a preamble to this invention.

### Lymph

A clear liquid that becomes slightly whitish or yellow when loaded with fat. It is composed of 90% water and 10% proteins, fats, dead cells, bacteria and malignant cells if any type of cancer is present.

Lymph comes from interstitial fluid (the fluid found in the spaces surrounding cells and originating from substances that leak from blood capillaries, which are the smallest type of blood vessel). Lymph helps bring oxygen and nutrients to cells and removes waste from them. As new interstitial fluid is formed, it replaces old fluid, which drains into the lymphatic vessels. When it enters the lymphatic vessels, the interstitial fluid is called lymph.

The function of lymph is to return proteins and excess interstitial fluid to the bloodstream. It can collect bacteria and deposit them in the lymphatic nodes, which destroy them.

Lymph has a viscosity comparable to that of blood plasma. The difference between the two is that lymph contains white blood cells; their purpose is to provide cells with water and nutrients, while collecting waste products from those cells along the way. Lymph enters the lymphatic vessels from the interstitial spaces.

The difference between the cardiovascular system and the lymphatic system is that the latter is not closed and does not have a central pump; transport is slow and sporadic with low pressure. Lymphatic movement occurs due to the natural contraction of the stomach and intestines, valves and compression during contraction of adjacent skeletal muscle and arterial pulsation.

Lymph is very similar in viscosity to blood. Its pressure, speed and circulation depend largely on the movements of the body's joints, muscles, and even human breathing.

### Lymph Nodes

These are small, flattened organs with a shape similar to a small kidney (reniform) that are arranged in chains along the path of the lymphatic vessels. Their size varies greatly, depending essentially on their degree of activity.

Nodes vary in size, the smallest measuring approximately 1 mm and the largest measuring between 1-2 cm, and are found in the armpits, groin, along the large vessels of the neck and in large numbers in the chest and abdomen.

The lymph nodes filter the lymph that passes through them. Lymph flows slowly through the sinuses and the reticular filaments that cross them acting as a mechanical filter, stopping or slowing the passage of infectious microorganisms and other cells. Lymph usually passes through at least two lymph nodes on its way from the tissues to the blood.

The lymph nodes are interspersed along the path of the lymphatic vessels. They have a functional structure that allows crossover of antigen-presenting cells, lymph-antagonistic substances and lymphocytes. Lymph nodes play a key role in initial immune response.

Through radioisotope lymphangiography, lymph node capacity has been measured at an average of 0.4 ml in volume of lymph.

### Lymphatic Vessel

These are responsible for carrying the lymph that forms in the tissues to the venous system, where it empties, specifically at the base of the neck, where the internal jugular and subclavian veins join to form the venous angle. The lymphatic vascular system adapts to the amount of lymph that is formed. If this increases, the frequency and amplitude of the lymphatic contractions also increase; thus, the lymphatic vascular system acts as a safety valve or compensating mechanism when the balance of Starling forces is altered. To better understand this invention, we will now cover the existing treatments that are commonly called “conventional treatments,” which describe different types of treatments that are currently performed on patients with lymphedema by specialists in the area, and their advantages and disadvantages.

### Mechanical Treatments

Elevation of the affected limb. Although elevation can be helpful, reducing lymphedema through the principle of gravity, it is often impractical and uncomfortable.
Elastic compression. Elastic compression between 20 to 60 mm Hg pressure is an effective measure for managing lymphedema and can be used as a primary therapy. The patient must wear an elastic sleeve from the wrist to the armpit. It should be replaced every 4 to 6 months or when it begins to lose elasticity. It has some drawbacks, including the discomfort associated with compression, it is relatively difficult to put on and, lastly, it is expensive. Professionals who prescribe these types of accessories must instruct patients on their proper application. In this regard, this task is often left to an employee of a medical supply store who lacks specific training.
Exercises. Moderate active exercises for the arm and forearm muscles can be used to activate the “muscle pump,” in the hope that this will increase venous and lymphatic flow. Exercises are best performed with minimal limb movement so that the patient can do them moderately when desired. Excessive repetition of exercises can cause an exaggerated increase in arterial blood flow to the limb, which can increase the formation of edema.
Manual lymphatic drainage. This is a massage technique that stimulates the lymphatic vessels to make them contract more frequently, and anatomically follows the lymphatic pathways of the body. Lymphatic drainage massages begin centrally in the neck and trunk with the aim of emptying the main lymphatic pathways, thus facilitating the drainage of lymph accumulated in the affected upper limb.
Bandages. Multi-layered bandages applied with low pressure may be used immediately after manual lymphatic drainage. The bandaged patient is then guided through exercises involving an active range of motion with the muscles and joints that function within the enclosed space of the bandage.
Pneumatic compression pumps. This has been proposed as an alternative to lymphatic massage. There are a variety of device models, but are basically of two types: some that use a single chamber and other more modern ones that have multiple chambers. The single chamber system provides intermittent non-segmented compression along the entire treated arm. However, it can also cause retrograde lymph flow, which may cause increased lymphedema at the distal end of the arm. Those with multiple chambers can provide sequential compression distally to proximally, theoretically propelling the fluid ahead of its compression. It is difficult to compare the effectiveness of both methods as there is no consensus regarding the optimal pump pressure, duration and frequency of sessions.
Complete decongestive physiotherapy. This therapeutic procedure takes into account that lymphedema affects a complete quadrant of the body, although its effects are more relevant in the arms and hands. It includes skin care, manual lymphatic drainage, multi-layered low-pressure compression bandages (followed by a tight compression garment when the edema has decreased), and therapeutic exercises with these garments or bandages. It should be performed by specialized and specially-trained therapists.
Physiotherapy treatments with bandages for lymphedema secondary to breast cancer have resulted in patients reducing the volume of the affected limb, as long as it involves cases of mild lymphedema.

### Drug Treatments

Antibiotics. From a pharmacological point of view, the most important thing is early treatment of erysipelas or lymphangitis with penicillin G sodium. It is also useful to administer delayed-release penicillin over long periods to prevent infectious outbreaks, which are of significant relevance in the progressive worsening of lymphedema.
Diuretics. These are not effective in reducing protein-rich edema such as lymphedema. Although they can temporarily mobilize water, the increase in oncotic pressure exerted by the high concentration of proteins in the interstitial spaces induces rapid reproduction of lymphedema and could even accelerate the fibrosis process.
Benzopyrones. Some benzopyrones and plant derivatives could improve chronic lymphedema through the activation of proteolysis, removing excess protein in tissue spaces leading to a decrease in oncotic pressure and edema fluid.
Skin lubricants. Of particular importance is keeping the skin of the affected limb well lubricated using protective emulsions.

### Surgical Treatment

Surgery has only been considered in cases where all conservative measures have failed. Numerous procedures have been proposed, but their results have been poor. In general, there are 2 types of surgical methods: physiological and reductive.
Physiological procedures: these aim to restore lymphatic flow to the limb by anastomosing the dilated lymphatic ducts with veins using microsurgical techniques.
Reduction techniques simply resect the excess edematous tissue to bring the limb to a more functional size. These procedures involve the removal of skin and subcutaneous tissue, followed by skin grafting. More recently, in Sweden, good short-term results have been reported with liposuction, although the procedure's long-term efficacy is not yet known.

In the case of surgical treatment, the main objectives in the management of lymphedema are to limit patient morbidity and improve functionality and quality of life. Surgical treatment is an alternative when conservative measures are no longer sufficient. There are procedures that seek to prevent the development of lymphedema and techniques that include physiological (reconstructive) and receptive procedures that are used when lymphedema is already established. Success depends on proper patient selection and individualized treatment.

### Innovative Treatments

The International Lymphedema Framework (ILF) is a platform made up of professionals, patients and companies in the sector created to research and disseminate updated information on lymphedema and the pathologies associated with it.
Margaret McNeely (Associate Professor in the Department of Physical Therapy at the University of Alberta in Edmonton, Canada) spoke about Decongestive Progressive Resistive Exercise (resistance exercise using compression garments in a sequence of decongestive exercises) highlighting the strong evidence supporting the benefits of exercise in cancer survivors for improved quality of life. The presentations dedicated to lymphedema in cancer patients focused on breast cancer. The possibility of cooling the skin to 10 degrees Celsius before manual lymphatic drainage was explained, achieving, according to preliminary observations, a decrease in fibrotic tissue and a “softer” consistency of the edema.
On the other hand, a very interesting study by Cestari provided data on the use of ultrasound for diagnosis and for choosing the most appropriate treatment strategy in patients with lymphedema in the upper limbs. The morphology and vascularization of the lymph nodes in the area, the structural characteristics of the edematous tissues, etc., are studied, obtaining a lot of relevant and useful information for treatment.
J. P. Belgrade explained the possibility of early detection of secondary lymphedema in cancer treatments through infrared fluorescence, with images obtained in both limbs, before and after lymph node removal.

In summary, research related to lymphedema as a consequence of cancer is aimed at early detection and the immediate reduction of symptoms in an attempt to provide the patient with the highest quality of life.

US 2021/01137523 A1, dated May 13, 2021, relates to devices and methods for performing a lymphovenous bypass procedure. A first ring is attached to tissue connected to at least one lymphatic channel of a patient and a second ring is attached to a vein of the patient. One end of the lymphatic channel extending through the first ring is inserted into an open end of the vein and the rings are connected to each other to establish the flow of fluid from the lymphatic channel to the vein; that is, it refers to the connection made between a lymphatic vessel and a vein. This procedure is carried out by means of 2 tips that are connected. In the invention that is claimed, the connection is made from the vessel to the lymph, making it possible to interconnect pieces to grow said connection, which is necessary for the lymph to continue functioning.

US 2017/0172765 A1, dated Jun. 22, 2017, mentions that the invention provides for a three-dimensional bioprinter for manufacturing cellular constructs such as tissues and organs using electromagnetic radiation (EMR) at 405 nm or higher. The bioprinter includes a material deposition device comprising a cartridge for receiving and containing a biomaterial-containing composition that cures after exposure to EMR.

The bioprinter also includes an EMR module that emits EMR at a wavelength of approximately 405 nm or higher. A bioprinter cartridge containing cells and a material curable at a wavelength of approximately 405 nm or higher is also provided. The cells are present in a chamber and are extruded through a hole to form the cellular construct. The above invention relates to protecting a bioprinter that is able to recreate organs and tissue. Although it is true at this point the lymphatic vessel is included, it is also true that neither the printer nor the software are protected, but rather the form that is created to reconnect the damaged lymphatic vessel with the lymph and that does not have a shape similar to the original one of the body, in addition to having adaptations enabling the forms to be assembled together repeatedly up to “n” number of times depending on the length necessary to replace the damaged duct.

US 2014/0012407 A1, dated Jan. 9, 2014, which mentions a bioprinter comprising: one or more printer heads, wherein a printer head comprises a means for receiving and holding at least one cartridge, and wherein said cartridge comprises contents selected from one or more of: bioink and support material; a means for calibrating the position of at least one cartridge; and a means for dispensing the contents of at least one cartridge. This document also describes methods for manufacturing a tissue construct, comprising: a computer module that receives the input of a visual representation of a desired tissue construct; a computer module that generates a series of commands, wherein the commands are based on the visual representation and are readable by a bioprinter; a computer module that provides the series of commands to a bioprinter; and the bioprinter deposits bioink and support material according to the commands to form a construct with a defined geometry. The intent of this patent is the same as the previous one, namely that what was intended to be protected is an invention relating to the bioprinter and processes for printing organs and tissues, which are not patentable subject matter within the present invention. In this regard, what they intend to protect is how to create various products. In the case of the present invention, what is intended to be protected is the final product, not the manufacturing process, which is mentioned solely as a reference to understand the type of final product in terms of shape, functionality, assembly and characteristics thereof, all of which are necessary to enable introduction into the human body without damage or side effects.

Finally, document US 2007/0129788 A1 was published on Jun. 7, 2007, with number, which relates to a series of venous valves that can be positioned in the vein, in order to achieve a connection from point A to point B; this system could be similar in terms of the connection. However, using a conduit with multiple connections is different from using a series of valves, where the valves themselves, by their nature, only regulate the flow of the conduit. In this sense, they support the vein but do not restore the damaged conduit, that is, the part of the lymphatic vessel itself.

In this regard, the detailed description will highlight the novelty that the intended invention possesses in relation to these documents and other products known to date.

### BRIEF DESCRIPTION OF THE INVENTION

The present invention relates to an artificial lymphatic vessel for secondary lymphedema comprising a tubular body having at least one inlet end with a first diameter and at least one outlet end with a second diameter and at least one one-way valve disposed inside the tubular body in proximity to the at least one outlet end, wherein the at least one one-way valve comprises a sudden reduction of the first diameter to generate a third diameter, followed by an area of uniform reduction of the third diameter until reaching a discharge area with a fourth diameter, wherein the ratio between the first diameter and the fourth diameter is between approximately 2.94:1 to approximately 1.25:1.

### DETAILED DESCRIPTION OF THE FIGURES

The illustrative embodiment can be described with reference to the accompanying figures, whereby:

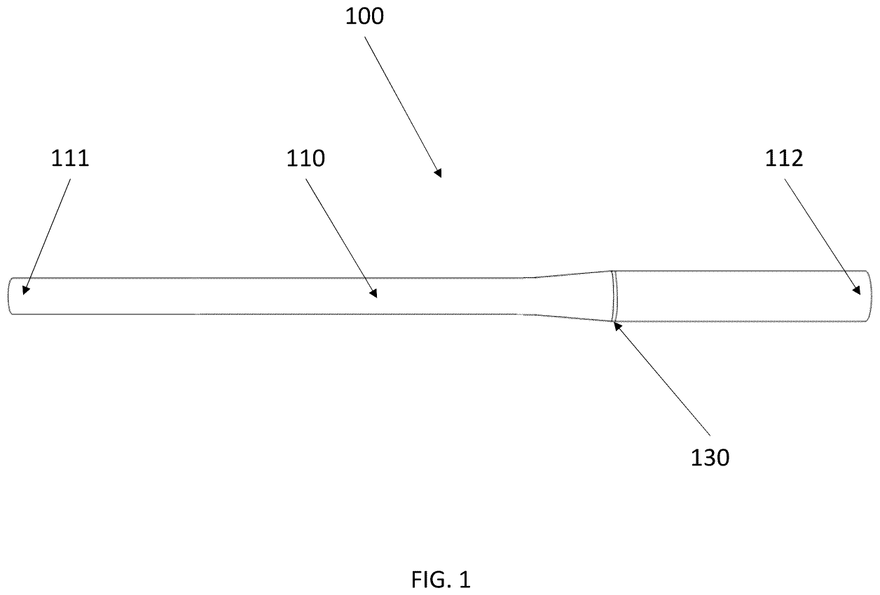

FIG. 1 is a side view of the artificial lymphatic vessel for secondary lymphedema of the present invention;

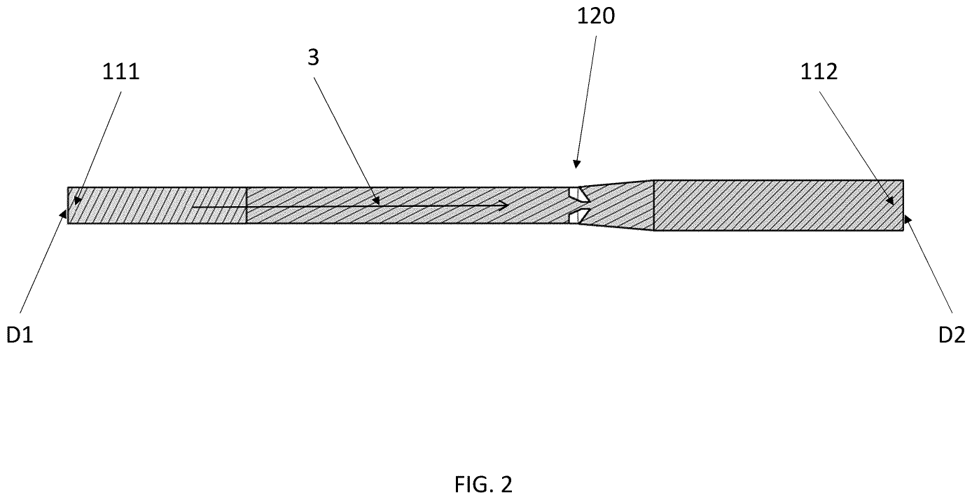

FIG. 2 is a longitudinal section of the artificial lymphatic vessel for secondary lymphedema of the present invention;

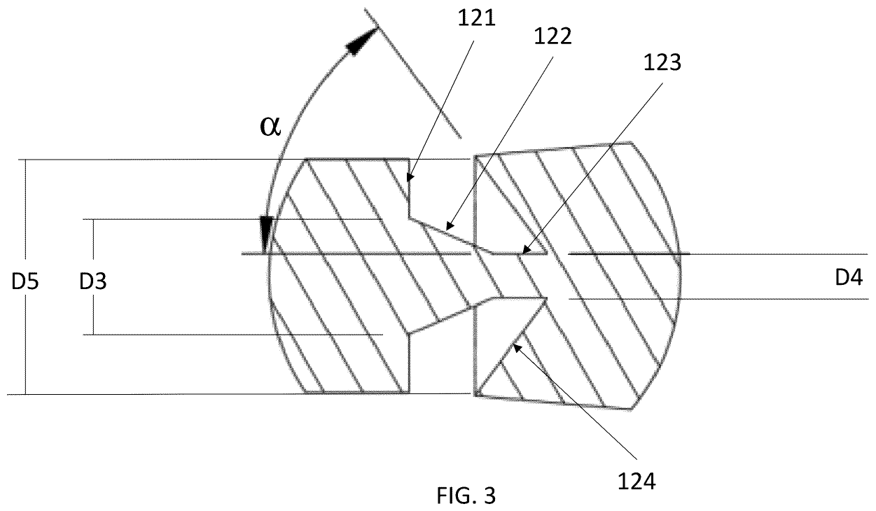

FIG. 3 is a detailed view of a one-way valve of the artificial lymphatic vessel for secondary lymphedema of the present invention;

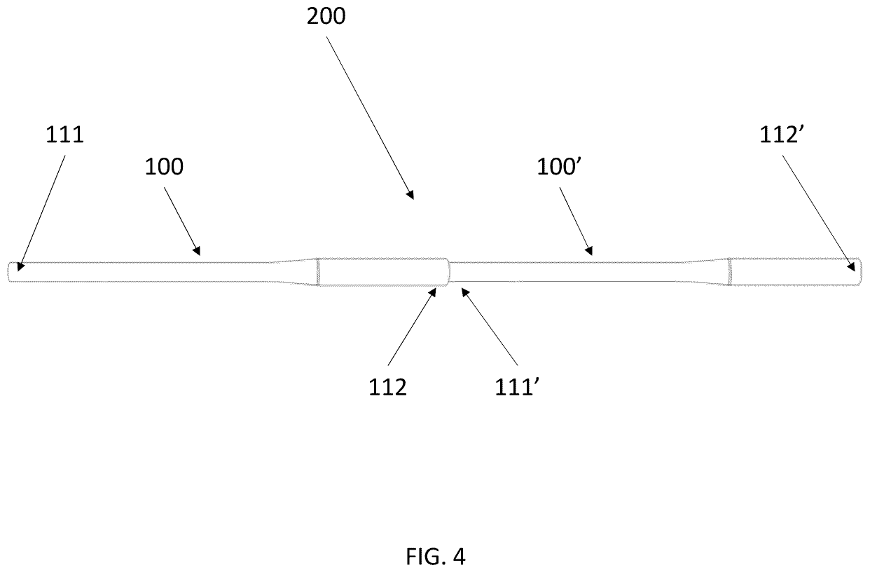

FIG. 4 is a side view of an artificial lymphatic vessel system of the present invention;

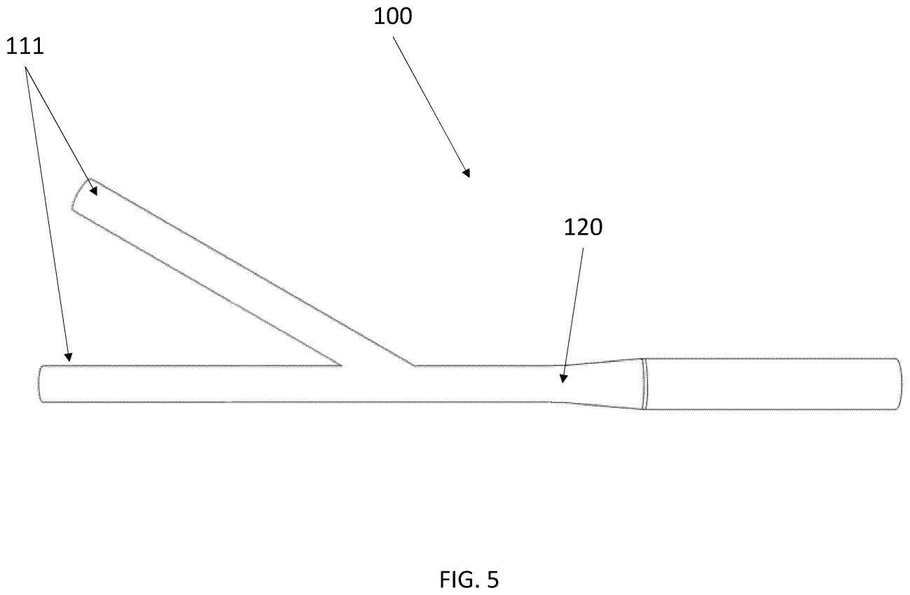

FIG. 5 is a side view of an alternative embodiment of the artificial lymphatic vessel for secondary lymphedema of the present invention;

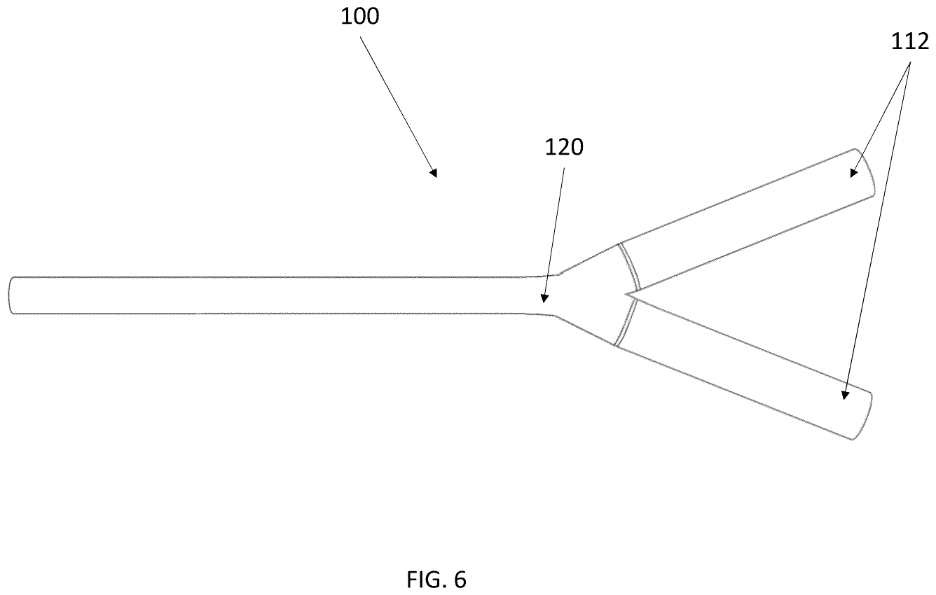

FIG. 6 is a side view of another alternative embodiment of the artificial lymphatic vessel for secondary lymphedema of the present invention;

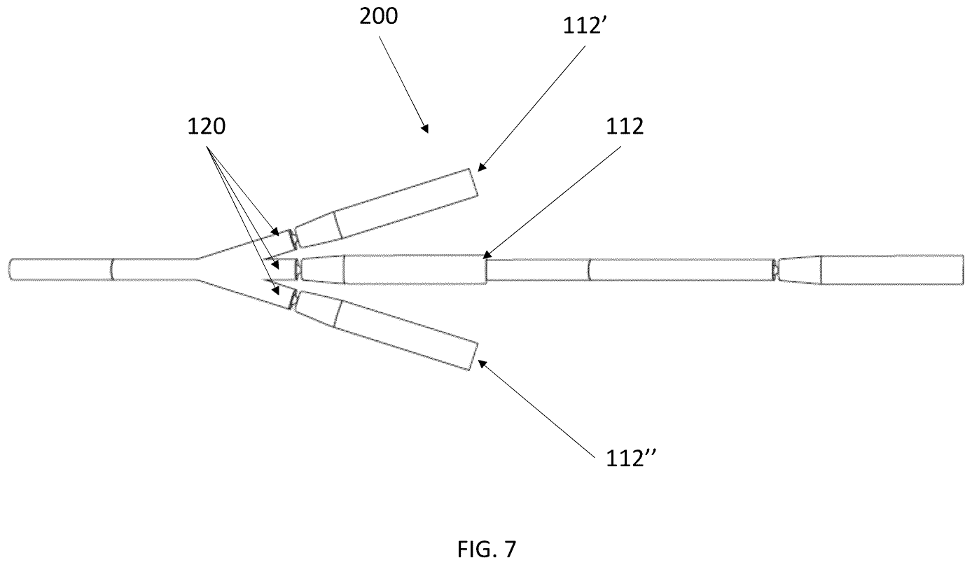

FIG. 7 is a side view of an alternative embodiment of the artificial lymphatic vessel system of the present invention;

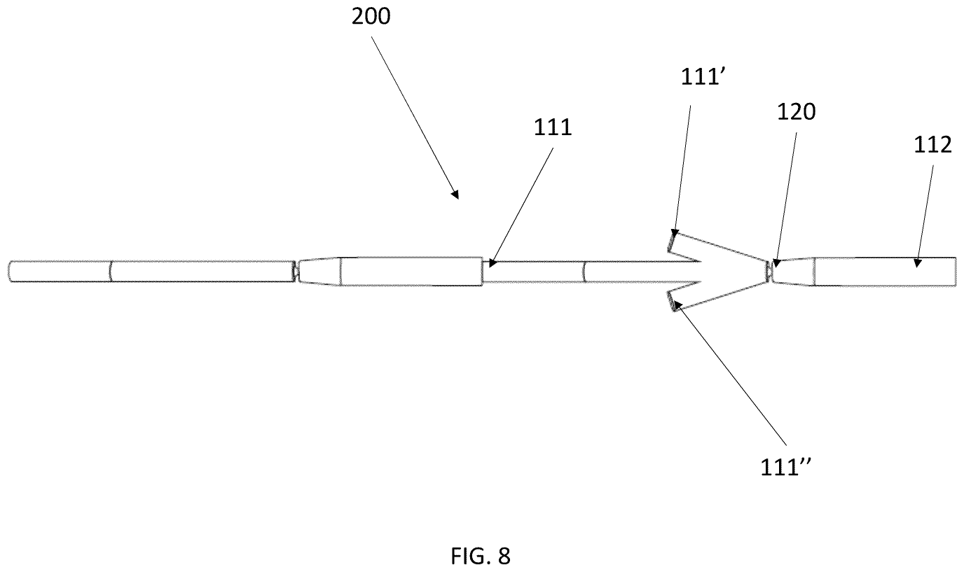

FIG. 8 is a side view of another alternative embodiment of the artificial lymphatic vessel system of the present invention.

### DETAILED DESCRIPTION OF THE INVENTION

The following detailed description is exemplary only and is not intended to limit the described embodiments or the application and uses of the described embodiments. As used herein, the word “exemplary” or “illustrative” means “serving as an example, instance, or illustration.” Any implementation described herein as “exemplary” or “illustrative” should not necessarily be construed as preferred or advantageous over other implementations. All of the implementations described below are exemplary implementations provided to enable those skilled in the art to make or use the embodiments of the disclosure and are not intended to limit the scope of the disclosure. In addition, there is no intention to be bound by any explicit or implicit theory presented in the technical field above, the background, the brief summary or the following detailed description. It should also be understood that the specific devices and processes illustrated in the accompanying drawings, and described in the following description, are merely exemplary embodiments of the inventive concepts defined in the attached claims.

The illustrations generally show non-limiting aspects of the systems and methods of the present description. The various aspects of the present descriptions of the devices should not be construed in any way as limiting the description. In addition, any modifications, concepts, and applications of aspects of the description are to be construed by those skilled in the art as comprised by, but not limited to, the illustrations and descriptions herein. Several modifications, equivalents, variants and alternatives, however, will be readily apparent to those skilled in the art. Any and all such modifications, variants, equivalents and alternatives are intended to fall within the spirit and scope of the present description.

As used herein, the singular articles “a”, “an”, and “the” include plural referents unless expressly and unambiguously limited to one referent. The term “proximal” refers to a direction toward the center or a central region of a device.

The term “distal” refers to an outward direction extending away from a central region of a device. However, it is to be understood that the description may assume several alternatives, variations, and sequences of steps, except where expressly stated otherwise. It is also to be understood that the specific devices and processes illustrated in the accompanying drawings and described in the following description are non-limiting representations of several aspects of the description. Therefore, the specific dimensions and other physical characteristics related to the aspects described herein are not intended to be and should not be considered as limiting.

Unless otherwise indicated, all ranges or ratios described herein are meant to encompass any and all sub-ranges or sub-ratios incorporated herein. For example, an established range or ratio of “1 to 10” should be considered to include any and all sub-ranges between (and including) the minimum value of 1 and the maximum value of 10; that is, all sub-ranges or sub-ratios that start with a minimum value of 10 or less, such as, but not limited to, 1 to 6.1, 3.5 to 7.8, and 5.5 to 10. Unless otherwise indicated, all numbers expressing dimensions, quantities of ingredients, flow rates, pressures, etc., used in the description and claims, are to be construed as modified in all instances by the term “approximately.” For the purposes of the present description, the term “approximately” determines a range of +10%, whereby if a value of “approximately 30%” is specified, the value is within a range of 27% to 33%.

The artificial lymphatic vessel (100) for secondary lymphedema of the present invention enables the restoration of lymph circulation to reach the lymph nodes in cases where reconstruction is not possible.

The basic functions will be to connect parts and dose substances, while the material must comply with specifications and regulations that ensure an implant is medical grade, similar to a real lymphatic vessel.

The artificial lymphatic vessel (100) for secondary lymphedema of the present invention is configured as modules so that the affected lymphatic vessels, depending on their length, can be replaced by the surgeon at the time of extraction. The material required for the lymphatic vessel to function must meet biocompatibility specifications and not be allergenic to the human body.

The artificial lymphatic vessel (100) for secondary lymphedema comprises a tubular body (110) having at least one inlet end (111) with a first diameter (D1) and at least one outlet end (112) with a second diameter (D2); wherein the second diameter (D2) is greater than the first diameter (D1) (D2>D1).

In a further configuration illustrated in FIG. 5, the tubular body (110) has two inlet ends (111, 111′) and one outlet end (112); in another further configuration illustrated in FIG. 6, the artificial lymphatic vessel (100) for secondary lymphedema has one inlet end (111) and two outlet ends (112, 112′); in another further configuration illustrated in FIGS. 7 and 8, the artificial lymphatic vessel (100) for secondary lymphedema has “n” inlet ends (111) and “n” outlet ends (112), “n” being an integer greater than or equal to 1.

Inside the tubular body (110) there is at least one unidirectional valve (120) near the at least one outlet end (112). Said at least one one-way valve (120) only allows the flow of fluid in one direction, indicated by arrow 3 in FIG. 2, such that the allowed backflow varies from a range of 0% to approximately 36%.

The at least one one-way valve (120) comprises a sudden reduction (121) of the first diameter (D1) to generate a third diameter (D3), followed by an area of uniform reduction (122) of the third diameter (D3) until reaching a discharge area (123) with a fourth diameter (D4); said area of uniform reduction (122) of the third diameter (D3) has a convergent truncated conical configuration.

After the at least one one-way valve (120), there is a sudden increase (124) in dimensions, which determines a fifth diameter (D5), followed by an area of uniform increase (125) in diameter until reaching said second diameter (D2), where the first diameter (D1) and the fifth diameter (D5) are equal (D1=D5); said area of uniform increase (125) in diameter has a divergent truncated cone configuration. Such sudden reductions and increases (121, 124) create turbulence inside the tubular body (110), which is desirable for the correct functioning of the artificial lymphatic vessel (100) for secondary lymphedema.

The uniform reduction area (122) of the third diameter (D3) is overlapped with the fifth diameter (D5), while the discharge area (123) is located in front of the fifth diameter (D5).

Additionally, the sudden increase in dimensions (124) occurs in the direction opposite to the flow of the fluid; said sudden increase in dimensions (124) presents an inclination a between approximately 61° and approximately 45° with respect to the horizontal, preferably with an inclination & of approximately 53°; in an alternative embodiment, said sudden increase in dimensions (124) has an angle of approximately 90°.

The backflow rate in the at least one one-way valve (120) is achieved by diameter ratios, where the ratio between the first diameter (D1), corresponding to said at least one inlet (111) and the fourth diameter (D4), corresponding to the discharge area (123), is between approximately 2.94:1 to approximately 1.25:1; in laboratory tests, for an inlet (111) with a first diameter (D1) of up to 500 microns (μm) the discharge area (123) has a fourth diameter (D4) that can range from 170 um up to 400 μm. Below the lower range indicated above, there is no flow of fluid through the at least one one-way valve (120), while above the upper range indicated above, there begins to be backflow of fluid through the at least one one-way valve (120).

The artificial lymphatic vessel (100) for secondary lymphedema has a circumferential channel (130) on its external surface in the area where the second diameter (D2) begins to indicate to the surgeon how far either a second artificial lymphatic vessel (100′) or a natural lymphatic vessel (not illustrated) can be introduced.

An artificial lymphatic vessel system (200) may comprise at least one pair of artificial lymphatic vessels (100, 100′) for secondary lymphedema, as described above, connected to each other, such that the at least one outlet (112) of a first artificial lymphatic vessel (100) for secondary lymphedema is coupled to the at least one inlet (111′) of a second artificial lymphatic vessel (100′) for secondary lymphedema and so on, to replace sections of natural lymphatic vessel. In an example where only two artificial lymphatic vessels (100, 100′) for secondary lymphedema were used, the at least one inlet (111) of a first artificial lymphatic vessel (100) for secondary lymphedema was adapted for connection with a natural lymphatic vessel, and the at least one outlet (112′) of the second artificial lymphatic vessel (100′) for secondary lymphedema is adapted for connection with a natural lymphatic vessel.

In the configurations illustrated in FIGS. 4, 5 and 6, the artificial lymphatic vessel (100) for secondary lymphedema has a one-way valve (120), regardless of the number of inlet ends (111) and outlet ends (112); while in the configurations illustrated in FIGS. 7 and 8, the artificial lymphatic vessel (100) for secondary lymphedema has matching one-way valves (120) with the same number of outlet ends (112, 112′, 112″), regardless of the number of inlet ends (111, 111′, 111″).

A person skilled in the art can modify the structure described herein. However, it should be noted that this description relates to preferred embodiments of the invention, and is provided for illustrative purposes only, and should not be understood as limiting the invention. All obvious modifications in the spirit of the invention, such as changes in the shape, material, and dimensions of the elements that make up the invention, should be considered within the scope of the attached claims.

1. An artificial lymphatic vessel for secondary lymphedema comprising: a tubular body having at least one inlet end with a first diameter and at least one outlet end with a second diameter; at least one one-way valve disposed inside the tubular body in proximity to the at least one outlet end; wherein the at least one one-way valve comprises a sudden reduction of the first diameter to produce a third diameter, followed by an area of uniform reduction of the third diameter until reaching a discharge area with a fourth diameter; wherein the ratio between the first diameter and the fourth diameter is between approximately 2.94:1 to approximately 1.25:1.

2. The artificial lymphatic vessel for secondary lymphedema according to claim 1, wherein the second diameter is greater than the first diameter.

3. The artificial lymphatic vessel for secondary lymphedema according to claim 2, wherein after the one-way valve, a sudden increase in dimensions occurs that determines a fifth diameter, followed by an area of uniform increase in diameter until reaching said second diameter.

4. The artificial lymphatic vessel for secondary lymphedema according to claim 3, wherein the first diameter and the fifth diameter are equal.

5. The artificial lymphatic vessel for secondary lymphedema according to claim 1, wherein the sudden reduction and the sudden increase in diameters have sharp edges.

6. The artificial lymphatic vessel for secondary lymphedema according to claim 1, wherein the area of uniform reduction of the third diameter has a convergent truncated conical configuration.

7. The artificial lymphatic vessel for secondary lymphedema according to claim 3, wherein the area of uniform diameter increase has a divergent truncated conical configuration.

8. The artificial lymphatic vessel for secondary lymphedema according to claim 3, wherein said sudden increase in dimensions occurs in the direction opposite the flow of the fluid.

9. The artificial lymphatic vessel for secondary lymphedema according to claim 8, wherein said sudden increase in dimensions has an inclination of between about 61° to about 45° with respect to the horizontal.

10. The artificial lymphatic vessel for secondary lymphedema according to claim 9, wherein said sudden increase in dimensions has an inclination of approximately 53°.

11. The artificial lymphatic vessel for secondary lymphedema according to claim 8, wherein the uniform reduction area of the third diameter overlaps with the fifth diameter.

12. The artificial lymphatic vessel for secondary lymphedema according to claim 9, wherein the discharge area is located in front of the fifth diameter.

13. The artificial lymphatic vessel for secondary lymphedema according to claim 1, wherein there is a circumferential channel on its external surface in the area where the second diameter begins.

14. The artificial lymphatic vessel for secondary lymphedema according to claim 1, wherein the number of outlet ends coincides with the number of one-way valves.

15. Artificial lymphatic vessel system comprising at least one pair of artificial lymphatic vessels for secondary lymphedema connected to each other according to claim 1, wherein the at least one outlet of a first artificial lymphatic vessel for secondary lymphedema is coupled to the at least one inlet of a second artificial lymphatic vessel for secondary lymphedema.

16. The artificial lymphatic vessel system according to claim 15, wherein the at least one inlet of the first artificial lymphatic vessel for secondary lymphedema is adapted for connection with a natural lymphatic vessel.

17. The artificial lymphatic vessel system according to claim 15, wherein the at least one outlet of the second artificial lymphatic vessel for secondary lymphedema is adapted for connection with a natural lymphatic vessel.
